(19) United States
(12) Patent Application Publication
(10) Pub. No.: US 2010/0204464 A1
(43) Pub. Date: Aug. 12, 2010

(54) ASCORBIC ACID DERIVATIVES, THEIR PREPARATION METHODS, INTERMEDIATES AND USES IN COSMETICS

(75) Inventors: Huashan Li, Nanjing (CN); Luqiu Shi, Nanjing (CN)

Correspondence Address: HUDAK, SHUNK & FARINE, CO., L.P.A., 2020 FRONT STREET, SUITE 307, CUYAHOGA FALLS, OH, 44221, US

(73) Assignee: NANJING ZHONGSHI CHEMICAL CO., LTD., Nanjing (CN)

(21) Appl. No.: 12/733,601

(22) PCT Filed: Sep. 14, 2007
(86) PCT No.: PCT/CN2007/002718

§ 371 Date: Mar. 10, 2010

(51) Int. Cl. C07H 1/00

(52) U.S. Cl. 536/123.1; 536/123.13

(57) ABSTRACT

A kind of ascorbic acid derivates 3-O-glyco-L-ascorbic acid, their preparation methods, intermediates and uses in cosmetics. The derivates used as vitamin C precursors have better physiological effect than 2-O-α-D-glucopyranosyl ascorbic acid (AA-2G) and are more stable. Present compounds can be used in many fields, such as in cosmetics, pharmaceuticals, foodstuffs and livestock feed, and especially as whitening agents in cosmetics. The preparation method involves protecting the 5,6-dihydroxyl of ascorbic acid, then coupling with 1-haloacylglycosyl, obtaining the intermediate 3-O-(acylglycosyl)-(5,6-O-isopropylidine)-L-ascorbic acid, removing the isopropylidine and acyl from the intermediates, thereby obtaining the target substance.

### TECHNICAL FIELD OF THE INVENTION

This invention relates to ascorbic acid derivatives, preparation method thereof and their intermediates as well as uses of the derivatives in cosmetics; in particular, relates to 3-O-glycosyl-L-ascorbic acid, preparation method thereof and its intermediate and use of said derivative in cosmetics.

### TECHNICAL BACKGROUND OF THE INVENTION

L-ascorbic acid, namely Vitamin C (abbreviated as VC), participates in many physiological activities in the body of human or animal. Since enzymes synthesizing ascorbic acid are lacking, Vitamin C can't be synthesized by human beings or animals themselves in their bodies and it must be provided by food, so it is listed in the essential nutrition elements of human beings or animals and plays an irreplaceable important role in the course of protecting human health and animal growth. In clinic, ascorbic acid is mainly used for preventing and curing scurvy and resisting infective diseases, promoting the healing of wound and fracture, and also used as an auxiliary element in medicines for treatment and health care. Lack of L-ascorbic acid may cause scurvy and result in the break of capillary with the sign of weak skin, loosened tooth and hemorrhagic gum as well as fragile skeleton. In addition to clinical applications, VC can be used as acidulant, reducing agent/antioxidant, bleaching agent and stabilizer, etc. in cosmetics, foods, medicines and feeds owing to its chemical structure and physiological activity. For example, it is served as the reducing agent, ultraviolet absorbent and melanin formation inhibitor in cosmetics. In actual animal raising, VC has such functions as synthesizing collagen, preventing fishes and shrimps from scurvy and black death, improving survival rate of the young, meeting the stress of animal and poultry as well as preventing abnormal bleeding and erosion of fish skeleton, etc.

However, as a water soluble vitamin, VC is extremely unstable in the aqueous solution and is easy to be destroyed by heat or resolved and oxidized by oxygen in the air and other oxidants. Especially, light, trace amount of heavy metallic element (such as Fe²⁺, Cu²⁺) and fluorescent material, etc. can promote it to be oxidized even more. The dehydrogenated ascorbic acid generated will be further oxidized or resolved into gulonic acid or other oxidation product without VC activity quickly and irreversibly; if VC is exposed to neutral pH, heat, light and heavy metal, it will degrade fast. This greatly restricts the application of VC. Therefore, how to increase the stability of ascorbic acid is the problem concerned about by domestic and international scholars at present. Since the seventies of the 20th century, people have been doing researches on different derivatives of ascorbic acid along with the hope of finding a new ascorbic acid derivative that can not only overcome the instability of ascorbic acid, but also exhibit better physiological functions of ascorbic acid.

The derivatives of ascorbic acid can be classified as salt, ester and saccharide derivatives of ascorbic acid. Saccharide derivative of ascorbic acid is a kind of important ascorbic acid derivative and many domestic and overseas literatures have reported various saccharide derivatives of ascorbic acid. People have chemically modified 2-, 3-, 5- and 6-hydroxyl of ascorbic acid through different biochemical synthesis or organic synthesis methods and synthesized many kinds of ascorbic acid derivatives. This kind of ascorbic acid derivative not only overcomes the shortcoming of general ascorbic acid, which is easy to be oxidized, but also can be absorbed and utilized well by human body and animal.

6-O-α-glucopyranosyl ascorbic acid (AA-6G) is the earliest found ascorbic acid derivative. As early as 1971, Suzuki et al. transferred the glucosyl group on maltose to ascorbic acid by using α-glucosidase which was generated from Aspergillus niger, but the concrete location of the glucosyl group was determined in recent years. Compared with ascorbic acid, AA-6G has a stronger stability and possesses reducing activity.

In addition, there is a kind of 5-O-α-D-glucopyranosyl ascorbic acid (AA-5G) that can be served as a booster of food quality and ultraviolet absorbent. In clinic, it can be used for preventing or treating infectious diseases such as virus diseases, bacterial diseases and malignant tumors. In cosmetics industry, it can be served as a skin repair agent and a whitener.

2-O-α-D-glucopyranosyl ascorbic acid (AA-2G) was jointly found by Hayashibara Biochemical Laboratories of Japan and the Department of Pharmacology of Okayama University and the method of synthesizing this Vitamin C derivative in quantity has been determined. This compound would not be oxidized since in the 2-position there is a glucose having masking function. AA-2G is very steady in aqueous solution and it has no direct reducibility in itself. AA-2G can be hydrolyzed by α-glucosidase at cell membrane while entering cell and the VC generated will be transported to the body and exhibit many physiological functions of VC in vivo. AA-2G can be synthesized by biotransformation method. It is safe, nonpoisonous and can be used as stabilizer, quality booster, physiological activator, ultraviolet absorbent and chemical and medical raw materials in food, beverage and medical industries. At present, AA-2G can only be produced by biotransformation method and the enzymes used are glycosyltransferases including α-glucosidase, α-cyclomaltodextrin glucanotransferase and α-amylase.

Suntory Ltd. of Japan further studied the β-isomer of AA-2G, i.e. 2-O-β-D-glucopyranosyl ascorbic acid, and prepared the same through chemical synthesis (J. Agric. Food Chem. 2004, 52, 2092-2096).

By further molecular modification of AA-2G, other derivatives 6-O-acyl-2-O-α-D-glucopyranosyl ascorbic acids can be obtained. These derivatives can improve the permeability of membrane and promote the effective transportation of the ascorbic acid derivatives. Said derivatives include: 6-butyryl-AA-2G, 6-caproyl-AA-2G, 6-capryloyl-AA-2G, 6-capryl-AA-2G, 6-dodecanoyl-AA-2G, 6-tetradecanoyl-AA-2G, 6-hexadecanoyl-AA-2G and 6-octadecanoyl-AA-2G. Studies indicated that the longer the chain of the acyl group is, the stronger the thermal stability of its molecule is and the stronger the ability of removing oxygen free radical is. Compared with other derivatives, said derivatives have remarkable advantages in removing oxygen free radical.

The formulas of the above mentioned saccharide derivatives of ascorbic acid are as follows:

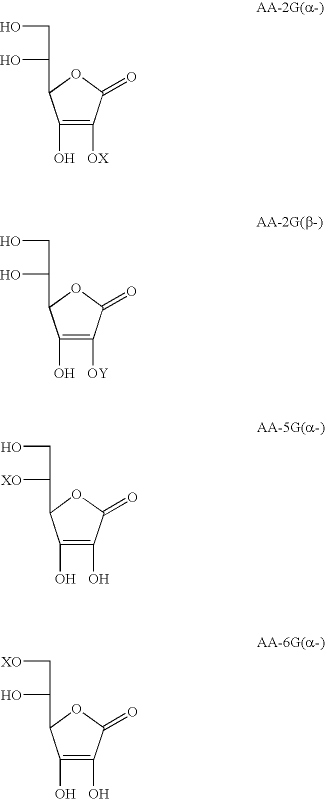

Wherein: X is α-glucoside and Y is β-glucoside. As derivatives of Vitamin C, the above compounds maintain certain VC activities and have improved stabilities compared with Vitamin C.

Few studies have been done on saccharide derivatives of ascorbic acid substituted at 3-O-position, moreover the saccharide in the derivative is only limited to monosaccharose; compared with other known saccharide derivatives of ascorbic acid, their stability is not obviously improved and their physiological activities have no superiority. Other ascorbic acid derivatives substituted by saccharide at 3-O-position have not been reported yet.

### SUMMARY OF THE INVENTION

The invention is to provide a new ascorbic acid derivative, more specifically, an ascorbic acid derivative, 3-O-glycosyl-L-ascorbic acid, with better stability, longer half life and more effective activities;

The invention is also to provide a synthesis method of 3-O-glycosyl-L-ascorbic acid;

The invention also provides an intermediate product 3-O-(acetyl glycosyl)-(5,6-O-iso-propylidene)-L-ascorbic acid, which is used for preparing 3-O-glycosyl-L-ascorbic acid;

The invention is also to provide an use of 3-O-glycosyl-L-ascorbic acid in cosmetics.

In this specification, the term “precursor of Vitamin C” refers to compounds that exhibit weak activities or have no activities of Vitamin C, but can be resolved to generate Vitamin C within the body or on the body surface of a human or an animal, or compositions containing the compounds.

The technical solution of the invention is as follows:

An ascorbic acid derivative having a structure of formula I:

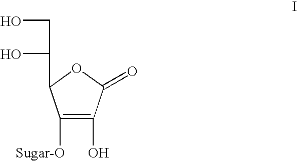

wherein said sugar is an oligosaccharide, or a biologically acceptable salt or an ester thereof.

In respect of chemical structure, sugar is an aldehyde derivative or a ketone derivative of polyhydric alcohol, including polyhydroxyaldehydes, polyhydroxyketones, polycondensates, or derivatives thereof. Said oligosaccharide may be formed through condensation of 2˜10 monosaccharide molecules, and can be hydrolyzed to monosaccharide molecules. Most common oligosaccharide is disaccharide, namely the saccharide formed by condensation of two monosaccharide molecules, such as maltose, isomaltose, lactose, gentiobiose, melibiose, cellobiose, chitobiose, N-acetamino lactose, etc.; the oligosaccharide may also be trisaccharide or tetrasaccharide (formed through condensation of three or four monosaccharide molecules), such as maltotriose, ginseng trisaccharide and acarbose, etc.; other oligosaccharides may also be used.

As a precursor of Vitamin C, said ascorbic acid derivative, 3-O-glycosyl-L-ascorbic acid has a better physiological function than 2-O-α-D-glucopyranosyl ascorbic acid (AA-2G), and a better stability than 2-O-α-D-glucopyranosyl-L-ascorbic acid (AA-2G), especially in its aqueous solution or a composition containing it. Just as other precursors of Vitamin C, such as AA-2G, said ascorbic acid derivative can be used in the field of cosmetics, quasi-medicines, medicines, foods and feeds.

The effects of preventing melanin from deposition (whitening effects) of 3-O-glycosyl-L-ascorbic acid and 2-O-α-D-glucopyranosyl-L-ascorbic acid (AA-2G) were evaluated by using B16F10 murine melanoma cells. With arbutin and kojic acid as positive control and on the basis of MTT experiment, high, middle and low concentrations (5.0 mM, 2.5 mM and 1.0 mM) were chosen to study the influence on the tyrosinase activity and melanin content in B16F10 murine melanoma cell (DOPA staining), and to compare the influences of samples on melanin synthesis. The experimental method comprising:

A. MTT experiment: to investigate the influence of each sample on the multiplication of B16F10 murine melanoma cell through cell culture.

B. Experiment for determining the activity of tyrosinase: to investigate through cell culture the influence of each sample on the activity of tyrosinase playing an important role in the formation of melanin.

C. Influence on the content of melanin: to qualitatively analyze the influence of each sample on the content of melanin in the system through DOPA staining.

D. Quantitative analysis of the content of melanin.

The results of the experiment indicate that:

1). Similar to kojic acid, when its concentration was higher than or equal to 5.0 mM, 2-O-α-D-glucopyranosyl-L-ascorbic acid (AA-2G) (VC derivative 1) significantly inhibited the multiplication of B16F10 cells; when its concentration was lower than 5.0 mM, it had no influence on the multiplication of B16F10 cells. Similar to arbutin, 3-O-lactosyl-L-ascorbic acid significantly inhibited the multiplication of B16F10 cells when its concentration was higher than or equal to 10.0 mM and had no influence on the multiplication of B16F10 cells when its concentration is lower than 10.0 mM.

2). 3-O-lactosyl-L-ascorbic acid (VC derivative 2) significantly inhibited the activity of tyrosinase at high, middle and low concentrations (5.0 mM, 2.5 mM and 1.0 mM). There was no statistical difference between three concentration groups of 3-O-lactosyl-L-ascorbic acid and arbutin, and no statistical difference between high concentration group of 3-β-lactosyl-L-ascorbic acid and kojic acid, but the functions of inhibiting the activity of tyrosinase of middle and low concentration groups of said derivative were weaker than those of kojic acid. At the same concentration, there was no statistical difference among the activities of 3-O-lactosyl-L-ascorbic acid, kojic acid and arbutin in respect of inhibiting melanin synthesis.

3). 2-O-α-D-glucopyranosyl-L-ascorbic acid (AA-2G) had a certain inhibiting effect on the activity of tyrosinase, but the effect was rather weaker than that of 3-O-lactosyl-L-ascorbic acid, and its effect of inhibiting the synthesis of melanin was relatively weak.

The stability research on 3-O-lactosyl-L-ascorbic acid indicated that it had a better stability than 2-O-α-D-glucopyranosyl-L-ascorbic acid (AA-2G), especially in aqueous solution or a formula. In the comparative experiment of stability between 3-O-lactosyl-L-ascorbic acid and 2-O-α-D-glucopyranosyl-L-ascorbic acid (AA-2G), both compounds were prepared as 10%, 5% and 1.0% aqueous solutions respectively and were placed at 0° C., 25° C. and 45V respectively for 3 months. Content analysis (HPLC, High Performance Liquid Chromatography) showed that at 0° C., two ascorbic acid derivatives were almost unchanged in contents and were both colorless; however, at 25° C. and 45° C., samples of 2-O-α-D-glucopyranosyl-L-ascorbic acid (AA-2G) all became obviously yellow and their contents all decreased; but samples of 3-O-lactosyl-L-ascorbic acid exhibited a rather good stability. Their solutions were all colorless and contents changed little. The experimental results of 3-O-lactosyl-L-ascorbic acid are shown in Table 1.

TABLE 1
Stability Experiment of 3-O-Lactosyl-L-Ascorbic Acid

| | Concentration % | | | | | |
|---|---|---|---|---|---|---|
| Temp- | 1% | | 5% | | 10% | |
| erature | Content | | Content | | Content | |
| °C. | (HPLC) | Color | (HPLC) | Color | (HPLC) | Color |
| 0 | 98 | Colorless | 98 | Colorless | 98 | Colorless |
| 25 | 98 | Colorless | 98 | Colorless | 98 | Colorless |
| 45 | 96 | Slight | 96 | Slight | 94 | Slight |
| | | color | | color | | color |

Notes:
the initial content of 3-O-lactosyl-L-ascorbic acid was 98% (HPLC).

Ascorbic acid derivative with the structure of Formula I has identical basic structure and similar properties with 3-O-lactosyl-L-ascorbic acid. As the precursor of Vitamin C, 3-O-glycosyl-L-ascorbic acid has a better physiological function and a better stability than 2-O-α-D-glucopyranosyl-L-ascorbic acid (AA-2G).

As a new precursor of Vitamin C, 3-O-glycosyl-L-ascorbic acid exhibits superior performance than 2-O-α-D-glucopyranosyl-L-ascorbic acid (AA-2G), such as the above mentioned effect of preventing melanin from deposition (whitening effects). Therefore, 3-O-glycosyl-L-ascorbic acid can be used in cosmetics.

As many known whiteners, 3-O-glycosyl-L-ascorbic acid can form many compositions and can be used in various cosmetics and skin care products, such as sunscreen products, anti-light ageing cosmetics, anti-crease cosmetics, etc.; it can be also extremely effective in keeping the elasticity of skin and inhibiting skin injury caused by ultraviolet ray. As required by the formulation of product, 3-O-glycosyl-L-ascorbic acid can be used in water and/or various organic solvents and can be used to prepare products with various additives, such as surfactant, thickener, pH modifier, preservative, softener, aromatic and/or perfume etc.; and can be made into liquid products or creams, etc.

The invention also provides a method of synthesizing 3-O-glycosyl-L-ascorbic acid, briefly, 5-hydroxyl and 6-hydroxyl of ascorbic acid are protected, then the ascorbic acid is coupled with 1-haloacyl sugar, and the product is obtained after deisopropylidene reaction and deacylation. A method of preparing ascorbic acid derivative with a structure of Formula I, comprising the steps of:

A) preparing 1-halogenated acylsaccharide: using saccharide as the raw material, acylating all hydroxyls of the raw material saccharide, and then halogenating to produce 1-halogenated acylsaccharide;

B) preparing intermediates: in presence of an alkali, producing the intermediate of 3-O-(acylglycosyl)-(5,6-O-isopropylidene)-L-ascorbic acid by condensating of 1-halogenated acylsaccharide and 5,6-O-isopropylidene-L-ascorbic acid.

C) removing protecting groups: removing isopropylidene and acyl by hydrolyzing of the intermediate produced in B) under acidic and alkaline conditions to produce 3-O-glycosyl-L-ascorbic acid.

The method of the invention will be described in detail below. The concrete reaction process is as follows:

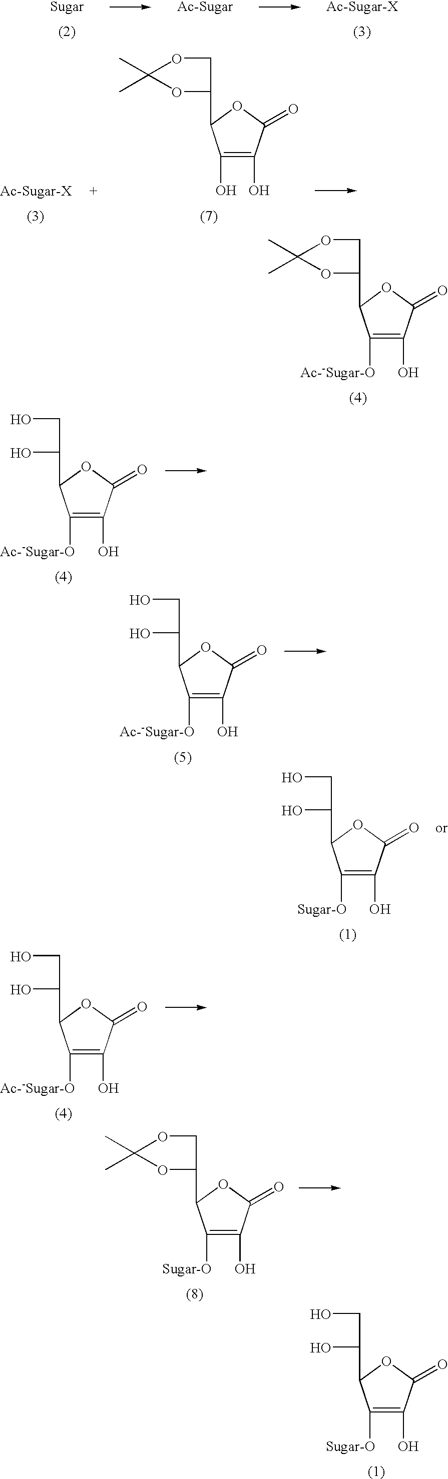

Sugar (2) is taken as raw material and omni-acylated and then halogenated to provide a 1-haloacyl sugar (3). The sugar (2) taken as raw material is oligosaccharide. The oligosaccharide can be disaccharide, such as maltose, isomaltose, lactose, gentiobiose, melibiose, cellobiose, chitobiose, N-acetamino lactose, etc.; the oligosaccharide can also be trisaccharide or tetrasaccharide, such as maltotriose, ginsengtrisaccharidea and acarbose, etc.; or other oligosaccharides. Halogen can be fluorine, chlorine or bromine; the protecting groups obtained from acylation reaction are common groups such as acetyl, propionyl, benzoyl or benzyl. For example, the hydroxyl in the raw material sugar (2) are all acetylated and then brominated to provide 1-bromo acetyl sugar (3) (Martors M. B., Preparation of acetorome-sugars, Naturem 1950m 165, 369).

Said 5,6-O-isopropylidene-L-ascorbic acid (7) in step B) can be prepared by technology known in the art. For example, 5,6-O-isopropylidene-L-ascorbic acid (7) may be obtained through condensation of L-ascorbic acid and acetone with L-ascorbic acid (6) as raw material and under the catalysis of acid (Chen H Lee, Paul A Seib, et al. Chemical synthesis of several phosphoric esters of L-ascorbic acid, Carbohydr Res, 1978. 67 (1), 127-135). The reaction process is shown below:

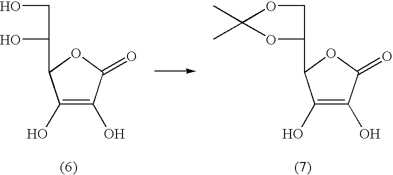

In 5,6-O-isopropylidene-L-ascorbic acid (7), 2-hydroxyl and 3-hydroxyl are exposed and 3-hydroxyl exhibits a certain acidity and can be coupled with 1-haloacyl sugar to form glucoside in a base environment to provide the intermediate product 3-O-(acylglycosyl)-(5,6-O-isopropylidene)-L-ascorbic acid (4). The reaction temperature is 0-100° C.; the solvent can be selected from methanol, ethanol, isopropanol, acetone or DMF. The acid generated in the reaction is absorbed by a base which can be an inorganic base, such as sodium carbonate, potassium carbonate, sodium bicarbonate, potassium bicarbonate, etc.; or an organic base, such as pyridine, triethylamine, etc. Unexpectedly, by using the method and the intermediate product of the invention, the resultant of this step was single, namely 3-O-(acylglycosyl)-(5,6-O-isopropylidene)-L-ascorbic acid and no 2-O-product was found. Thus, removal of the protecting group set forth in step C) can be carried out directly negating the need of purification.

The intermediate product 3-O-(acylglycosyl)-(5,6-O-isopropylidene)-L-ascorbic acid (4) is hydrolyzed in acidic and alkaline condition respectively to remove the protecting groups, i.e. isopropylidene and acyl to provide 3-O-glycosyl-L-ascorbic acid (1). Isopropylidene may be first removed by acid catalysis to provide 3-O-(acylglycosyl)-L-ascorbic acid (5), and then the resultant can be hydrolyzed in an alkaline condition to remove the protecting acyl group to provide the target product. Or, the sequence of removing the protecting groups may be different, the intermediate product is hydrolyzed in an alkaline condition first to remove acyl, and then isopropylidene is removed by acid catalysis to provide the target product.

Isopropylidene may be removed by acid catalysis from 3-O-(acylglycosyl)-(5,6-O-isopropylidene)-L-ascorbic acid (4) or 3-O-glycosyl-(5,6-O-isopropylidene)-L-ascorbic acid (8). The acid can be used includes: hydrochloric acid, sulfuric acid, phosphoric acid, p-toluene sulfonic acid, formic acid, acetic acid, trifluoroacetic acid, or propionic acid, etc. The solvent can be used includes: methanol, ethanol, acetone, or their aqueous solutions; Water can also be used; reaction temperature: 0-100° C.

The protecting acyl group may be removed by the hydrolysis of 3-O-(acylglycosyl)-(5,6-O-isopropylidene)-L-ascorbic acid (4) or 3-O-(acylglycosyl)-L-ascorbic acid (5) in alkaline condition. The base used may be aqueous solution of sodium hydroxide, potassium hydroxide, potassium carbonate, sodium carbonate, potassium bicarbonate and sodium bicarbonate or metal alcoholate, such as sodium methoxide or sodium ethoxide, etc. The solvent may be water, alcohol or aqueous solution of alcohol, such as methanol, ethanol or their aqueous solutions to dissolve the raw material, such as 3-O-(acylglycosyl)-L-ascorbic acid. The reaction temperature is 0-100° C. The reaction solution can be neutralized by using hydrochloric acid, sulfuric acid or cation exchange resin. In case hydrochloric acid or sulfuric acid is used, the salt generated is required to be removed. However, in case cation exchange resin is used, the step of removing the salt is not needed owing to the adsorption of sodium salt and potassium salt.

Through the above steps, organic solution or aqueous solution containing 3-O-glycosyl-L-ascorbic acid can be prepared. The solution may be lyophilized or the solvent may be removed through vacuum distillation to obtain the target compound.

As the precursor of Vitamin C, 3-O-glycosyl-L-ascorbic acid which is prepared according to the invention has a better physiological function than other saccharide derivatives of ascorbic acid, such as 2-O-α-D-glucopyranosyl ascorbic acid (AA-2G), and a better stability than other saccharide derivatives of ascorbic acid, such as 2-O-α-D-glucopyranosyl ascorbic acid (AA-2G); compared with other ascorbic acid derivatives, 3-O-glycosyl-L-ascorbic acid has the following advantages: no strong acidity, little skin stimulation; long effect and stability, slow release of Vitamin C in vivo and in vitro. 3-O-glycosyl-L-ascorbic acid can be used in the field of cosmetics, medicines, foods and feeds, etc., in particular used as a whitener in cosmetics.

According to the chemical synthesis method of 3-O-glycosyl-L-ascorbic acid of the invention, various ascorbic acid derivatives substituted by 3-O-glycosyl can be prepared pursuant to the various raw material sugars. Furthermore, the raw materials of this preparation method are easy to acquire and the method is simple with high yield.

The invention will be further described through examples below. The scope of the invention shall not be limited to the examples illustrated and described.

### DETAILED DESCRIPTION OF THE INVENTION

### Example 1

### Preparation of 1-bromo-hepta-O-acetyl-lactose (3a)

To a three-necked flask equipped with thermometer and dropping funnel, 180 mL of acetic anhydride was added and cooled to 0° C. in an ice-salt bath, 0.6 mL of perchloric acid was dropped slowly in the acetic anhydride and the internal temperature of the solution was controlled to 0-5° C.; after completion of adding the perchloric acid, the ice-salt bath was moved away. At room temperature, 50.0 g of anhydrous lactose was added in several batches, and the internal temperature of the solution was controlled to 33° C. After completion of adding the anhydrous lactose, the reacting solution was cooled to 10° C., 7.5 g of red phosphorus was put into the reacting solution, stirred to disperse the red phosphorus, then 14.5 mL of bromine was dropped into the reacting solution and the internal temperature of the solution was controlled below 20° C.; after completion of adding the bromine, 10.0 mL of ice-water was dropped and the temperature of the solution was controlled below 15° C.; after completion of dropping the ice-water, stirred for 2.0 h at room temperature, then the reacting solution was poured into ice-water, extracted with trichloromethane for several times; the organic phase was combined; dried with anhydrous magnesium sulfate, concentrated to obtain a yellow oil, then the yellow oil was dissolved in 75.0 mL of anhydrous ether, and preserved in a refrigerator overnight; a lot of white crystals appeared; the white crystals were filtered under reduced pressure, dried and 81.0 g of white solid powder was obtained the melting point of which was 123.0-124.5° C. The yield was 81.0%.

### Example 2

### Preparation of 5,6-O-isopropylidene-L-ascorbic acid (7)

To a dried 1 L three-necked flask, 91.0 g of ascorbic acid and 450 mL of acetone were added. The temperature was cooled to −5° C. in an ice-salt bath; 200.0 g of concentrated sulfuric acid was dropped slowly for approximately 2.5 hours and the internal temperature of the solution was controlled to 0-5° C., stirred for 5.0 min, then the ice-salt bath was moved away; the temperature was increased naturally to room temperature, and the reaction was continued for 45 minutes; the reacting solution changed from colorless to pale yellow; then the reacting solution was subjected to vacuum filtration under reduced pressure and the filter cake was washed for several times with a small amount of acetone until the pH value was neutral; the filter cake was dried at 50° C. in a vacuum for 1-2 hours and 89.5 g of white solid powder was obtained the melting point of which is 215-217° C. The yield was 80.2%.

### Example 3

### Preparation of 3-O-(hepta-O-acetyl-D-lactosyl)-(5,6-O-isopropylidene)-L-ascorbic acid (4a)

To a dried 1 L round-bottom flask, 79.0 g of 1-bromo-hepta-O-acetyllactose (3a), 28.1 g of 5,6-O-isopropylidene-L-ascorbic acid (7), and 500 mL of acetone were added, stirred to disperse; next 28.0 g of potassium carbonate, and 1.0 g of TEBAC were added, heated at 50° C. overnight and then was subjected to vacuum filtration; next, the solvent was recovered and a pale yellow oil was obtained; the pale yellow was dissolved with 200 mL of ethyl acetate, washed with 20 mL of saturated salt water for several times, dried with anhydrous sodium sulfate, ethyl acetate was recovered and then the residue was dried in vacuum by a pump for 1 hour, and 57.0 g of bubble-shaped yellow solid was obtained the melting point of which was 52.5-54.0° C. The yield was 60.2%.

¹HNMR (CDCl₃, 400M) δ: 1.21 (6H, —CH₃), 2.03-2.21 (21H, —CH₃), 3.98 (2H, —CH₂—), 4.32 (2H, —CH₂—), 4.37 (2H, —CH₂—), 4.45 (1H, —CH—), 4.47 (1H, —CH—), 4.49 (1H, —CH—), 4.50 (1H, —CH—), 4.52 (1H, —CH—), 4.54 (1H, —CH—), 4.61 (1H, —CH—), 4.65 (1H, —CH—), 4.68 (1H, —CH—), 5.91 (1H, —CH—), 5.58 (1H, —CH—), 5.73 (1H, —CH—);

MS (ESI, m/z): [M-H]⁻: 834.2

### Example 4

### Preparation of 3-O-(hepta-O-acetyl-D-lactosyl)-L-ascorbic acid (5a)

To a 500 mL round-bottom flask, 31.0 g of 3-O-(hepta-O-acetyl-D-lactosyl)-(5,6-O-isopropylidene)-L-ascorbic acid (4a), 180 mL of glacial acetic acid, and 180 mL of water were added, stirred to dissolve, the temperature was increased and maintained at 50-60° C. in an oil bath, and stirred for 1.5 h; TLC detecting was used to make sure that there was no point of the raw material, the solvent was recovered and the residue was dissolved with 250 mL of ethyl acetate and then washed with saturated salt water for several times. The organic phase was dried with anhydrous sodium sulfate and concentrated to obtain a pale yellow oil. The pale yellow oil was dried in vacuum for 1.0 hour at room temperature and 25.0 g of bubble-shaped yellow solid was obtained. The yellow solid was subjected to column chromatography and 22.1 g of bubble-shaped white substance was obtained, with a yield of 70.0%.

¹HNMR (CDCl₃, 400M) δ: 2.11-2.40 (21H, —CH₃), 3.68 (2H, —CH₂—), 4.31 (2H, —CH₂—), 4.43 (2H, —CH₂—), 4.48 (1H, —CH—), 4.53 (1H, —CH—), 4.55 (1H, —CH—), 4.61 (1H, —CH—), 4.64 (1H, —CH—), 4.68 (1H, —CH—), 4.71 (1H, —CH—), 4.75 (1H, —CH—), 4.89 (1H, —CH—), 5.22 (1H, —CH—), 5.38 (1H, —CH—), 5.46 (1H, —CH—);

MS (ESI, m/z): [M-H]⁻: 794.2

### Example 5

### Preparation of 3-O-(D-lactosyl)-L-ascorbic acid (1a)

At room temperature, 25.0 g of 3-O-(hepta-O-acetyl-D-lactosyl)-L-ascorbic acid (5a) was dissolved in 250 mL of methanol, 250 mL of 10% potassium carbonate water solution was then slowly added, stirred for 1.5 hours; next, cation exchange resin was added and the pH value was adjusted to 6.0-7.0; then filtered under reduced pressure and the filtrate was concentrated to obtain a yellow solid. The yellow solid was recrystallized and 6.1 g of a white or white-like solid was obtained, with a yield of 70.2%.

¹HNMR (D₂O, 400M) δ: 3.59 (2H, —CH₂—), 4.07 (2H, —CH₂—), 4.19 (2H, —CH₂—), 4.23 (1H, —CH—), 4.27 (1H, —CH—), 4.29 (1H, —CH—), 4.35 (1H, —CH—), 4.36 (1H, —CH—), 4.41 (1H, —CH—), 4.43 (1H, —CH—), 4.45 (1H, —CH—), 4.95 (1H, —CH—), 4.98 (1H, d, —CH—), 5.08 (1H, —CH—), 5.33 (1H, —CH—);

MS (ESI, m/z): [M-H]⁻: 500.1

### Example 6

The use of 3-O-(D-lactosyl)-L-ascorbic acid (1a) obtained from Example 5 in a whitening cream. Using 1.5 parts (by weight, the same below) of polyoxyethylene (25) lanolin alcohol ether and 2.5 parts of Monostearin as the emulsifying system, and 4 parts of cetosteryl alcohol, 5 parts of white petrolatum, and 5 parts of triglyceride caprylate decanoate (GTCC) as the main oil phase to prepare the base of O/W whitening cream, then adding 1-3 parts of 3-O-(D-lactosyl)-L-ascorbic acid in the anaphase of the cream emulsification (at approximately 45° C.).

### Examples 7-13

In Examples 7-13, different saccharides were used as raw materials to prepare 3-O-glycosyl-L-ascorbic acid containing different glycosyls in accordance with the methods of this invention.

The method of Example 1 was used for preparation of 1-bromo-acetylsaccharide (3b-3h);

The method of Example 3 was used for preparation of 3-O-(acetylglycosyl)-(5, 6-0-isopropylidene)-L-ascorbic acid (4b-4-h);

The method of Example 4 was used for preparation of 3-O-(acetylglycosyl)-L-ascorbic acid (5b-5h);

The method of Example 5 was used for preparation of 3-O-glycosyl-L-ascorbic acid (1b-1 h).

See Table 2 for mole yields of the obtained target products and intermediates:

TABLE 2
Mole Yields of Various Products and Intermediates (%)

| | | Intermediates or Products | | |
|---|---|---|---|---|
| | | Yields (%) | | |
| Structure of Sugar (2) | | 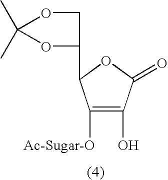 | 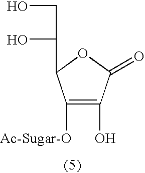 | 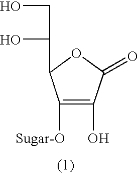 |
| (2a)* | 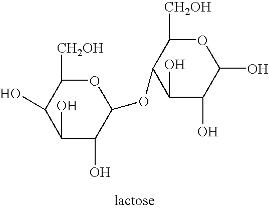 | 60.2 | 70 | 70.2 |
| (2b) | 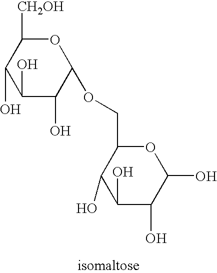 | 59 | 68 | 76 |
| (2c) | 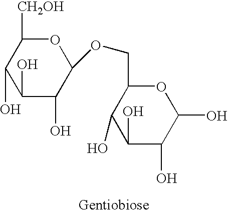 | 63 | 69 | 61 |
| (2d) | 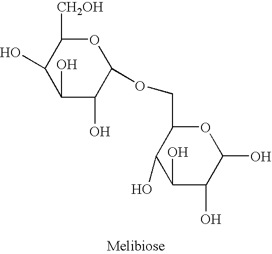 | 69 | 76 | 79 |
| (2e) | 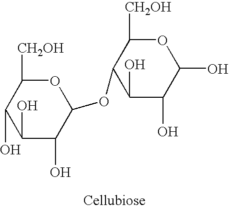 | 55 | 72 | 68 |
| (2f) | 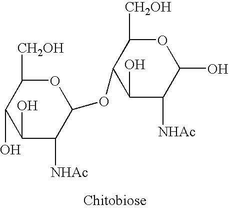 | 49 | 55 | 59 |
| (2g) | 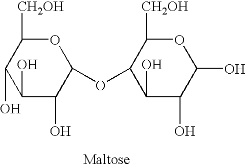 | 67 | 61 | 82 |
| (2h) | 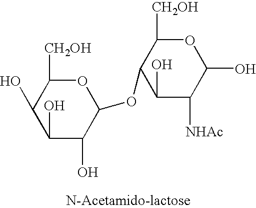 | 65 | 65 | 61 |

*Data of lactose (2a) are the yields of Examples 3-5.

Various 3-O-glycosyl-L-ascorbic acids so obtained may be used as the active whitening substance to replace 3-O-(D-lactosyl)-L-ascorbic acid in a whitening cream in accordance with the method of Example 6.

### Examples 14

All cells were seeded in the 96-well plate respectively at a density of 1*10⁴/well, and incubated under 5% CO₂ and at 37° C. for 24 h, then the supernatant was removed and to each well was added 2004 culture medium containing a certain concentration of medicine. Three concentrations, high, middle and low, were set for each medicine, each concentration in quadruplicate wells. For control group, 200 μL culture medium was directly added and incubated for 72 h. 20 μL MTT solution (5 g/L) was added to each well and the sample was incubated under 5% CO₂ and at 37° C. for 4 h, then the supernatant was removed and to each well was added 150 μL DMSO. The sample was vibrated for 10 min and the absorbance of each well was measured at a wavelength of 490 nm (reference wavelength: 620 nm) by enzyme-label instrument. Cell multiplication rate=(average absorbance of each concentration of medicine to be screened)/(average absorbance of control group)*100%.

Results are shown in Table 3.

TABLE 3
Multiplication rate of melanoma cell under the influence of different
concentration of medicine measured by MTT method (Mean ± SE, %)

| | Concentration | | | |
|---|---|---|---|---|
| Medicine | 20.0 mM | 10.0 mM | 5.0 mM | 1.0 mM |
| VC derivative 1 | 8.17 ± 0.46** | 43.21 ± 7.40** | 100.98 ± 14.79 | 108.66 ± 9.32 |
| VC derivative 2 | 66.67 ± 5.48* | 76.56 ± 7.67 | 79.34 ± 3.99 | 72.67 ± 10.59 |
| Kojic acid | 43.55 ± 7.36** | 43.10 ± 2.31** | 63.23 ± 3.66 | 92.20 ± 8.28 |
| Arbutin | 46.15 ± 3.41* | 76.94 ± 6.08 | 102.94 ± 3.64 | 113.57 ± 5.57 |

SPSS11.0 software and one-way ANOVA were utilized in data statistics. The multiplication rate of melanoma cells of blank control group was 100%.
*p < 0.05,
**p < 0.01

### Example 15

B16F10 cells were seeded in the 96-well plate respectively at a density of 5*10³/well, and incubated under 5% CO₂ and at 37° C. for 24 hour, then the supernatant was removed and to each well was added 100 μL culture medium containing different concentration of medicine to be screened and for blank control group, only culture medium was added. Each group was in quadruplicate and the culture medium was renewed every other day. After being incubated for 6 days, each well was washed by PBS without Ca²⁺ and Mg²⁺, and then 100 μL Triton-X solution (0.5%) was added to each well. After being vibrated in ultrasound for 30 minutes, 50 μL L-DOPA solution (10 mM/L) was added to each well and the samples was kept at 37° C. for 3 hours. The absorbance of each well was measured at a wavelength of 490 nm (reference wavelength: 620 nm) by enzyme-label instrument. Influence rate of the activity of tyrosinase=(average absorbance of the group of medicine to be screened)/(average absorbance of control group)*100%

Results are shown in Table 4.

TABLE 4
Activity of tyrosinase of B16F10 melanoma cell under the influence
of different concentration of medicine (Mean ± SE, %)

| | Concentration | | |
|---|---|---|---|
| Medicine | 5.0 mM | 2.5 mM | 1.0 mM |
| VC derivative 1 | 69.90 ± 1.733** | 92.40 ± 0.94 | 95.99 ± 2.01 |
| VC derivative 2 | 20.68 ± 1.34** | 36.19 ± 2.98** | 51.01 ± 2.71** |
| Kojic acid | 18.32 ± 1.12** | 23.00 ± 2.11** | 36.74 ± 3.22** |
| Arbutin | 20.53 ± 1.02** | 38.07 ± 1.78** | 50.02 ± 4.04** |

SPSS11.0 software and one-way ANOVA were utilized in data statistics. The tyrosinase activity of melanoma cells of control group was 100%.
*p < 0.05,
**p < 0.01

### Example 16

According to the results of MTT experiment, B16F10 cells were seeded in the 6-well plate respectively at a density of 2*10⁴/well, and incubated under 5% CO₂ and at 37° C. for 24 h, then the supernatant was removed and to each well was added 6.0 mL culture medium containing different concentration of medicine to be screened and for blank control group, only culture medium was added. Each group was in quadruplicate and the culture medium was renewed every other day. After being incubated for 6 days, each well was washed by PBS twice, fixed by 4% paraformaldehyde for 15 min, and then washed by PBS. After being incubated in 0.5% L-DOPA at 37° C. for 0.5 h, the well was taken a picture under microscope (10*10)

Comparison of the photos of VC derivatives 2 with the blank control group, we can significantly see lower degree of staining, indicating that VC derivatives 2 could significantly inhibit the activity of tyrosinase, reduce melanin production.

### Example 17

B16F10 cells were seeded in φ 60 mm culture mediums, and incubated under 5% CO₂ and at 37° C. for 24 h, then the supernatant was removed and to each medium was added culture medium containing different concentration of medicine and for control group, only culture medium was added. Each group was in triplicate and the culture medium was renewed every other day. After being incubated for 6 days, the cells were digested by 0.25% trypsin/EDTA and collected, then washed by PBS twice. The cells of each group were counted and 0.2 mL redistilled water was added to make the cells suspended for 1 min respectively, then the mixture of 500 μL ethanol and 500 μL ether was added. The cells were placed at room temperature for 15 min and centrifuged at a rate of 3000 rpm for 5 min, then the supernatant was removed and 1 ml NaOH (1 mol/L, containing 10% DMSO) was added to the precipitate. After being placed at 80° C. for 30 min, 4 mL redistilled water was added to dilute the concentration of NaOH to 0.2 mol/L. The absorbance of each group was measured at a wavelength of 475 nm (reference wavelength: 620 nm) by spectrophotometer. The content of melanin=[(absorbance of the group of medicine to be screened/average amount of cells)/(average absorbance of control group/average amount of cells)]*100%

Results are shown in Table 5.

TABLE 5
Influence of different medicine on the content
of melanin of B16F10 cells (Mean ± SE, %)

| | Concentration | | |
|---|---|---|---|
| Medicine | 5.0 mM | 2.5 mM | 1.0 mM |
| VC derivative 1 | 84.19 ± 1.85 | 91.11 ± 1.82 | 96.37 ± 3.63 |
| VC derivative 2 | 22.24 ± 1.80** | 30.08 ± 3.77** | 51.01 ± 2.71** |
| Kojic acid | 17.65 ± 1.42** | 24.15 ± 1.88** | 37.20 ± 4.33** |
| Arbutin | 20.53 ± 1.02** | 36.61 ± 2.15** | 50.12 ± 1.97** |

SPSS11.0 software and one-way ANOVA were utilized in data statistics. The content of melanin of blank control group was 100%.
*p < 0.05,
**p < 0.01

### Example 18

### Preparation of 3-O-(D-lactosyl)-L-ascorbic acid (1a)

11.0 g of 3-O-(hepta-O-acetyle-D-lactosyl)-(5,6-O-isopropylidene)-L-ascorbic acid (4a), 90 mL of glacial acetic acid, and 90 mL of water were added to a 500 mL round-bottom flask, then was stirred to dissolve; the temperature was increased and maintained to 50-60° C. in an oil bath, stirred for 1.5 h, TLC detecting was used to make sure that there was no point of the raw material; the solvent was recovered, and the residue was dissolved with 100 mL of methanol, 100 mL of 10% of potassium carbonate water solution was added slowly, stirred for 40 min, then a cation exchange resin was added; the pH value was adjusted to 6.0-7.0; then filtered under reduced pressure and the filtrate was concentrated to obtain a yellow solid. The yellow solid was recrystallized and 2.3 g of a white or white-like solid was obtained, with a yield of 35.2%.

### Example 19

### Preparation of 3-O-(D-lactosyl)-L-ascorbic acid (1a)

At room temperature, 11.0 g of 3-O-(hepta-O-acetyl-D-lactosyl)-(5,6-O-isopropylidene)-L-ascorbic acid (4a) was dissolved with 250 mL of methanol, 100 mL of 25% of potassium carbonate water solution was added slowly, stirred for 1.5 h, then a cation exchange resin was added; the pH value was adjusted to 6.0-7.0; then filtered under reduced pressure and the filtrate was concentrated to obtain a yellow oil. Next, 80 mL of glacial acetic acid and 80 mL of water was added, stirred to dissolve, the temperature was increased and maintained at 50-60° C. in an oil bath, stirred for 1.5 h; TLC detecting was used to make sure that there was no point of the raw material; the solvent was recovered and a yellow oil was obtained. The yellow oil was dried for 1.0 h in a vacuum at room temperature and then recrystallized to obtain 1.95 g of a pale yellow solid, with a yield of 29.6%.

### Example 20

### Preparation of 3-O-(D-lactosyl)-L-ascorbic acid (1a)

At room temperature, 5.0 g of sodium methanol (50%) was dissolved with 250 mL of methanol, then 25.0 g of 3-O-(hepta-O-acetyl-D-lactosyl)-L-ascorbic acid (5a) was added; stirred for 2.0 h; a cation exchange resin was added; the pH value was adjusted to 6.0-7.0; then filtered under reduced pressure and the filtrate was concentrated and 10.9 g of a white solid was obtained, with a yield of 69.6%.

Examples 21-24 are for Preparation of 3-O-(hepta-O-acetyl-D-lactosyl)-(5,6-O-isopropylidene)-L-ascorbic acid (4a).

### Example 21

By following the method of Example 3, and using sodium carbonate as the alkali to obtain a pale yellow solid.

### Example 22

Following the method of Example 3, using methanol as the solvent, and pyridine as the alkali.

### Example 23

Following the method of Example 3, using ethanol as the solvent, and triethylamine as the alkali.

### Example 24

Following the method of Example 3, using DMF as the solvent, and sodium bicarbonate as the alkali.

Examples 25-28 are for Preparation of 3-O-(hepta-O-acetyl-D-lactosyl)-L-ascorbic acid (5a)

### Example 25

Following the method of Example 4, using hydrochloric acid as the acid, and methanol as the solvent.

### Example 26

Following the method of Example 4, using p-phenylacetic acid as the acid, and methanol-water solution as the solvent.

### Example 27

Following the method of Example 4, using p-toluenesulfonic acid as the acid, and ethanol-water solution as the solvent.

### Example 28

Following the method of Example 4, using phosphoric acid as the acid, and acetone-water solution as the solvent.

### Example 29

### Preparation of 3-O-(D-lactosyl)-L-ascorbic acid (1a)

Following the method of Example 5, using sodium ethanol as the alkali, and anhydrous ethanol as the solvent.

### Example 30

In accordance with the methods of this invention, prepare 3-O-glycosyl-L-ascorbic acid containing maltotriosyl, wherein:

Following the method of Example 1 for preparation of 1-bromo-acetylsaccharide;

Following the method of Example 3 for preparation of 3-O-(acetylglycosyl)-(5,6-O-isopropylidene)-L-ascorbic acid (4b-4-h);

Following the method of Example 4 for preparation of 3-O-(acetylglycosyl)-L-ascorbic acid;

Following the method of Example 5 for preparation of 3-O-glycosyl-L-ascorbic acid.

### Example 31

In accordance with the methods of this invention, prepare 3-O-glycosyl-L-ascorbic acid containing panoxtriosyl, wherein:

Following the method of Example 1 for preparation of 1-bromo-acetylsaccharide;

Following the method of Example 3 for preparation of 3-O-(acetylglycosyl)-(5,6-O-isopropylidene)-L-ascorbic acid (4b-4-h);

Following the method of Example 4 for preparation of 3-O-(acetylglycosyl)-L-ascorbic acid;

Following the method of Example 5 for preparation of 3-O-glycosyl-L-ascorbic acid.

### Example 32

In accordance with the methods of this invention, prepare 3-O-glycosyl-L-ascorbic acid containing maltotriosyl, wherein:

Following the method of Example 1 for preparation of 1-bromo-acetylsaccharide;

Following the method of Example 3 for preparation of 3-O-(acetylglycosyl)-(5,6-O-isopropylidene)-L-ascorbic acid (4b-4-h);

Following the method of Example 4 for preparation of 3-O-(acetylglycosyl)-L-ascorbic acid;

Following the method of Example 5 for preparation of 3-O-glycosyl-L-ascorbic acid.

1. An ascorbate derivative with the structure shown in Formula I: 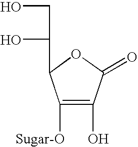
wherein said sugar is an oligosaccharide, or bio-acceptable salt or ester thereof.

2. The ascorbate derivative according to claim 1, wherein said oligosaccharide is a di-, tri-, or tetra-saccharide.

3. The ascorbate derivative according to claim 2, wherein said oligosaccharide is maltose, isomaltose, lactose, gentiobiose, melibiose, cellobiose, chitobiose, or N-acetyllactosamine.

4. A method for preparing the ascorbate derivatives according to claim 1, comprising:
A) preparing 1-halogenated acylsaccharide: using saccharide as the raw material, acylating all hydroxyls of the raw material saccharide, and then halogenating to produce 1-halogenated acylsaccharide;
B) preparing intermediates: in presence of an alkali, producing the intermediate of 3-O-(acylglycosyl)-(5,6-O-isopropylidene)-L-ascorbic acid by condensating of 1-halogenated acylsaccharide and 5,6-O-isopropylidene-L-ascorbic acid;
C) removing protecting groups: removing isopropylidene and acyl by hydrolyzing of the intermediate produced in B) under acidic and alkaline conditions to produce 3-O-glycosyl-L-ascorbic acid.

5. The method according to claim 4, wherein said saccharide is maltose, isomaltose, lactose, gentiobiose, melibiose, cellobiose, chitobiose, or N-acetyllactosamine.

6. The method according to claim 4, wherein for step A), first acetylating the raw material saccharide and then brominating the resultant to produce 1-bromoacetylsaccharide.

7. The method according to claim 4, wherein said 5,6-O-isopropylidene-L-ascorbic acid of Step B) is produced by condensation of L-ascorbic acid and acetone under catalysis of an acid.

8. The method according to claim 4, wherein for step B), the reaction temperature is 0-100° C., using methanol, ethanol, isopropyl alcohol, acetone, or DMF as the solvent, and sodium carbonate, potassium carbonate, sodium bicarbonate, or potassium bicarbonate, or pyridine or triethylamine as the alkali.

9. The method according to claim 4, wherein for Step C), isopropylidene is removed under catalysis of an acid, the reaction temperature being 0-100° C., using hydrochloric acid, sulfuric acid, phosphoric acid, p-toluenesulfonic acid, formic acid, acetic acid, trifluoroacetic acid, or propionic acid as the acid, and water, methanol, ethanol, acetone, or their water solution as the solvent.

10. The method according to claim 3, wherein for step C), the acyl group is hydrolyzed and removed under alkaline conditions, the reaction temperature being 0-100° C., using sodium hydroxide, potassium hydroxide, potassium carbonate, sodium carbonate, potassium bicarbonate, or sodium bicarbonate, or sodium methanol or sodium ethanol as the alkali, and water, methanol, ethanol, or their water solution as the solvent.

11. An intermediate 3-O-(acylglycosyl)-(5,6-O-isopropylidene)-L-ascorbic acid for producing said ascorbate derivatives according to claim 1.

12. Use of said ascorbate derivatives according to claim 1 in cosmetics.
